(19) United States
(12) Patent Application Publication
(10) Pub. No.: US 2008/0021179 A1
(43) Pub. Date: Jan. 24, 2008

(54) CATALYTIC CHEMICAL REACTION PROCESS

(76) Inventors: Wilhelmus Petrus Mul, Amsterdam (NL); Josephus Johannes Hermanus Rijsemus, Amsterdam (NL)

Correspondence Address: SHELL OIL COMPANY, P O BOX 2463, HOUSTON, TX, 772522463, US

(21) Appl. No.: 11/770,236

(22) Filed: Jun. 28, 2007

(30) Foreign Application Priority Data
Jun. 30, 2006 (EP) 06253440.9

(51) Int. Cl. C08F 4/26; C07C 41/20

(52) U.S. Cl. 526/90; 568/695

(57) ABSTRACT

The present invention relates to a catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream is separated from the process stream in at least one separation zone, and wherein one or more magnetic filters are employed to remove at least a portion of the solid possessing ferromagnetic properties from the process stream without removal or deactivation of the homogeneous catalyst present in the process stream.

### FIELD OF THE INVENTION

The present invention relates to a catalytic chemical reaction process, wherein the chemical reaction process is performed using a homogeneous catalyst based upon cobalt, nickel or iron, and wherein one or more magnetic filters are employed to remove from the process and/or product stream at least a portion of solids possessing ferromagnetic properties formed as a degradation product of the homogeneous catalyst.

### BACKGROUND OF THE INVENTION

Several important industrial chemical reactions are catalysed using a homogeneous catalyst based on cobalt, nickel or iron. Illustrative, but not exhaustive, examples of such industrial chemical reactions that may be catalysed using a homogeneous catalyst based on cobalt, nickel or iron are hydroformylation, carbonylation, and olefin oligomerisation and polymerisation reactions.

It is known that most catalysts have a finite lifetime, typically due to poisoning of the catalyst by impurities/by-products present, or due to the catalyst becoming degraded by temperature or pressure. Catalyst degradation products are typically present in any industrial chemical reaction process stream which uses a homogeneous catalyst. Catalyst degradation products often have different catalytic properties than the original catalyst. For instance, catalyst degradation products may be catalytically inactive, or in some cases, they may catalyse competing reactions, either of the original chemical reactant, or of the desired chemical product.

Catalyst degradation products may remain homogeneous within the process stream, however, in many cases the catalyst degradation product present in the process stream comprises a solid. If the homogeneous catalyst is based upon cobalt, nickel or iron, the solid product which is formed by the catalyst degradation may possess ferromagnetic properties.

Because catalysts are often an expensive component in continuously operated industrial chemical reactions, it is desirable to recycle at least a portion of the active catalyst. However, if the degraded catalyst does not catalyse the desired reaction, and may even catalyse undesirable side reactions, it would be desirable to remove at least some of the catalyst degradation products. Another problem that can be experienced in homogeneously catalysed chemical reaction systems, is that the presence of catalyst degradation products can promote further degradation of the active catalyst present.

Furthermore, the solid formed as a product of the catalyst degradation, can cause excessive wear on pumps and seals in the chemical reactor system. As a result of this increased wear on pumps and seals, the chemical reactor system requires increased maintenance to replace and repair the pumps and seals, frequently involving complete shutdown of the reactor system. Such maintenance is highly undesirable since it increases the operational running costs of the reactor, and also reduces the annual chemical output of the reactor. This is a particular problem in continuously operated chemical reaction systems, wherein a stream containing the homogeneous catalyst (and also a solid degradation product of the catalyst) is recycled from the product separation process back to the reactor.

Therefore, when catalytic chemical reactions are catalysed by a homogeneous catalyst based on cobalt, nickel or iron, and the degradation product of the homogeneous catalyst comprises a solid possessing. ferromagnetic properties, it would be desirable to remove the solid from the process stream of the catalytic chemical reaction.

GB-A-0,670,423 describes a method of reducing the concentration of dissolved cobalt, nickel or iron catalytic metals from liquid chemical reaction products formed by the reaction of organic compounds with CO and H₂ operated at superatmospheric pressure, which comprises reducing the partial pressure of carbon monoxide in order to precipitate the metal from the liquid product and separating the metal from the liquid using a magnetic field. However, such a process removes active homogeneous catalyst from the liquid product, and as such would only be suitably applied to chemical product streams, and thus does not solve the problems associated with the presence of solids produced by the degradation of homogeneous catalysts being present in process streams of catalytic chemical reactions.

### SUMMARY OF THE INVENTION

The present invention relates to a catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream is separated from the process stream in at least one separation zone, and wherein one or more magnetic filters are employed to remove at least a portion of the solid possessing ferromagnetic properties from the process stream without removal or deactivation of the homogeneous catalyst present in the process stream.

The present invention also relates to a catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream comprising at least 50 wt % organic compounds, a homogeneous catalyst based upon cobalt, nickel or iron, and a degradation product of the homogeneous catalyst comprising a solid possessing ferromagnetic properties, is separated from the process stream in at least one separation zone, and wherein one or more magnetic filters are employed to remove at least a portion of the solid possessing ferromagnetic properties from the product stream without removal or deactivation of the homogeneous catalyst present in the product stream.

The present invention also relates to a process for the hydroformylation of an olefinic compound having at least one carbon-to-carbon double bond to produce an alcohol and/or aldehyde, comprising the steps of:
(i) contacting the olefinic compound with carbon monoxide and a source of hydrogen in the presence of a homogeneous hydroformylation catalyst based on cobalt, to produce a process stream, the process stream additionally comprising a degradation product of the homogeneous catalyst comprising a solid possessing ferromagnetic properties;
(ii) separating a product stream comprising an alcohol and/or aldehyde from the process stream; and
(iii) removing at least a portion of the solid possessing ferromagnetic properties from the process stream using at least one magnetic filter.

The present invention also relates to a process for the oligomerisation of at least one olefinic monomer to produce an olefinic oligomer having at least one carbon-to-carbon double bond, comprising the steps of:
(i) contacting at least one olefinic monomer with a homogeneous oligomerisation catalyst based on cobalt, nickel or iron, to produce a process stream, the process stream additionally comprising a degradation product of the homogeneous catalyst comprising a solid possessing ferromagnetic properties;
(ii) separating a product stream comprising an olefinic oligomer from the process stream; and
(iii) removing at least a portion of the solid possessing ferromagnetic properties from the process stream using at least one magnetic filter.

The present invention further relates to the use of a magnetic filter in a homogeneous catalytic chemical reaction process to remove at least a portion of solids possessing ferromagnetic properties from a process and/or product stream without removal or deactivation of the homogeneous catalyst present in the process and/or product stream.

### DETAILED DESCRIPTION OF THE INVENTION

As used herein, the term “magnetic filter” is used to mean any unit which generates a magnetic field. The magnetic filter is used in the process of the present invention to remove solids possessing ferromagnetic properties from a process or product stream comprising at least 50 wt % organic compounds, a homogeneous catalyst based upon cobalt, nickel or iron, and a degradation product of the homogeneous catalyst comprising a solid possessing ferromagnetic properties. In one embodiment, the process stream to which the magnetic filtration is to be applied is a flowing process stream. Typically, the magnetic filter comprises at least one magnetic element, the magnetic field of which protrudes into the process or product stream to which the magnetic filtration is to be applied.

In one embodiment, the magnetic filter generates a magnetic field from a magnetic element located in at least one wall of the vessel containing the process or product stream to which the magnetic filtration is to be applied. As used herein, the term “vessel” means any piece of apparatus which contains a process and/or product stream, e.g. pipe-work, reactors, separators and the like. Optionally, the magnetic filter may be arranged in such a manner that it can be removed from the vessel and reintroduced to the vessel in order to facilitate cleaning and/or maintenance of the magnetic filter.

In another embodiment, the magnetic filter generates a magnetic field from at least one element which protrudes from at least one wall of the vessel containing the process and/or product stream to which the magnetic filtration is to be applied. Optionally, the magnetic filter may be arranged in such a manner that it can be removed from the vessel and reintroduced to the vessel in order to facilitate cleaning and/or maintenance of the magnetic filter. Typically, in this embodiment of the magnetic filter, the magnetic field is generated by multiple elements protruding from at least one wall of the vessel containing the process and/or product stream.

In a further embodiment, the magnetic filter generates a magnetic field from at least one element which is suspended within the vessel containing the process and/or product stream to which the magnetic filtration is to be applied. The magnetic filter is typically suspended within the vessel by employing a suitable holder located within the vessel. Optionally, the magnetic filter may be arranged in such a manner that it can be removed from the vessel and reintroduced to the vessel in order to facilitate cleaning and/or maintenance of the magnetic filter. Typically, in this embodiment, the magnetic field is generated by multiple elements suspended within the vessel containing the process and/or product stream.

In yet another embodiment, the magnetic filter comprises a magnetic field generated from any combination of the methods described above.

The source of the magnetic field used in the magnetic filter is not critical and may be generated by any known method. For example, the source of the magnetic field may be a permanent magnet or a switchable magnet. Examples of suitable permanent magnets include, but are not limited to, rare earth, ferrite, ceramic and alnico magnets. An example of a suitable switchable magnet includes, but is not limited to, an electromagnet.

The magnetic filter employed in the present invention may be any commercially available magnetic filter or may be bespoke to fit specific requirements of the specific chemical reaction system and/or reactor series for which it is to be employed.

In one embodiment of the present invention, the magnetic filter is arranged such that it may be removed from the process and/or product stream to enable cleaning. The exact configuration used to enable the magnetic filter to be removed from the process and/or product stream for cleaning is not critical and varies depending upon the configuration of the apparatus containing the process and/or product stream. Suitable configurations would be easily determined by the person skilled in the art. The exact method of cleaning the magnetic filter is not critical and would be dependent upon the nature of the captured solids possessing ferromagnetic properties and/or the nature of the magnetic filter. Suitable cleaning processes would be known to the person skilled in the art. For example, the magnetic filter can be cleaned by any suitable chemical or physical methods. An example of a chemical method of cleaning the magnetic filter can be dissolving the captured solids possessing ferromagnetic properties in a suitable solvent/liquid, such as an acid or other reactive liquid. Examples of physical methods of cleaning the magnetic filter include scraping the captured solids possessing ferromagnetic properties off the magnetic element of the magnetic filter, or by removing the captured solids possessing ferromagnetic properties from the magnetic element of the magnetic filter by applying a centrifugal force, a hydrodynamic force or other similar force. Alternatively, or in addition to the above-disclosed cleaning methods, the cleaning of the magnetic filter can be performed by removing the magnetic field from the magnetic filter. This can readily be achieved by using a switchable magnet, or by removing the magnetic element from the magnetic filter.

By the term “process stream”, as used herein, it is meant any stream which feeds the chemical reactor series (inlet process stream), is used internally within the chemical reactor series (internal process stream) or is resultant from the chemical reactor series (outlet process stream).

By the term “product stream”, it is meant a stream which is separated from the process stream and comprises at least one product compound generated by the catalytic chemical reaction process. The concentration of the at least one product compound in the product stream is greater than the concentration of the at least one product compound in the process stream.

The process stream of the present invention comprises at least 50 wt % organic compounds, a homogeneous catalyst based upon cobalt, nickel or iron, and a degradation product of the homogeneous catalyst comprising a solid possessing ferromagnetic properties. Preferably, the process stream of the present invention comprises at least 60 wt % organic compounds, more preferably at least 70 wt % organic compounds and especially at least 75 wt % organic compounds. In one embodiment of the present invention, the process stream comprises at most 99.99999 wt % organic compounds, preferably 99.9999 wt % organic compounds.

The process stream comprises at least one compound which is liquid under the conditions of the reaction zone. Preferably the compound which is liquid under the conditions of the reaction zone is an organic compound.

Examples of organic compounds that may be present in the process and/or product stream of the present invention include inert gaseous organic compounds, dissolved inert gaseous organic compounds, reactant compounds, product compounds, co-catalyst compounds, promoters, stabilisers, secondary catalysts, solvents and by-products of the chemical reaction process.

Examples of inorganic compounds that may be present in the process and/or product stream of the present invention include inert gaseous inorganic compounds, dissolved inert gaseous inorganic compounds, inorganic reactant compounds, in organic product compounds, inorganic catalyst compounds, co-catalyst compounds, promoters, stabilisers, secondary catalysts, inorganic solvents (e.g. water) and inorganic by-products of the chemical reaction process.

Preferably, at least one reactant and at least one product of the chemical reaction process is organic.

The composition of the process stream varies depending upon the particular stage of the reaction system. For example, before the reaction zone, the concentration of reactants is much greater than the concentration of products; after the reaction zone but before the product separation zone, the concentration of products increases relative to the concentration of products before the reaction zone; after the separation zone, the concentration of the products decreases relative to the concentration of products after the reaction zone but before the product separation zone.

By the term “homogeneous catalyst” as used herein, it is meant that the active catalyst used in the chemical reaction is completely miscible with the process stream. The homogeneous catalyst used in the present invention comprises a metal selected from cobalt, nickel or iron, or any combination thereof. The homogeneous catalyst typically comprises other additional components, said additional components would be known to a person skilled in the art of homogeneous catalysis and may include components based upon metals other than cobalt, nickel or iron, as well as components not containing metals.

In the process and/or product stream to which the magnetic filter is applied there is present the degradation product of the homogeneous catalyst. In the present invention, the degradation product of the homogeneous catalyst comprises a solid which possesses ferromagnetic properties. The solid which possesses ferromagnetic properties typically forms a suspension of fine particles in the process stream, but may also form larger particles or agglomerate to form larger particles. In one embodiment, the catalyst degradation product comprising a solid which possesses ferromagnetic properties is not catalytically active for the same chemical reaction as the active homogeneous catalyst.

The catalytic chemical reaction process of the present invention is performed in a chemical reactor series which comprises at least one chemical reaction zone and at least one separation zone. Typically, the chemical reactor series comprises: at least one inlet; at least one reaction zone; at least one separation zone; and, at least one chemical product outlet. Optionally, the chemical reactor series may also comprise a waste stream outlet.

If the catalytic chemical reaction is to be performed in a continuous fashion, the chemical reactor series may also comprise a recycle loop for recycling at least a portion of the process stream back to the at least one reaction zone. The recycled process stream typically contains at least the active homogeneous catalyst based on cobalt, nickel, iron or a combination thereof, and an organic liquid component which may comprise valuable unreacted chemical reactants and/or a solvent, and/or the chemical reaction product, which may act as a solvent.

The at least one inlet of the chemical reactor series is used to feed the components of the process stream to the at least one reaction zone. Commonly, the chemical reactor series comprises more than one inlet. Typically, the chemical reactor series comprises from 1 to 10 inlets. Each inlet in the chemical reactor series may be used to introduce a single component of the process stream or several components of the process stream. In some instances, the homogeneous catalyst comprising cobalt, nickel or iron may be introduced in the form of discrete constitutional components via several inlets and the active homogeneous catalyst is then formed in-situ.

The number of reaction zones is not critical, but typically there would be between 1 and 20 reaction zones. The term “reaction zone” as used herein means a controlled environment which contains the process stream, and wherein the chemical process may occur. A reaction zone can be, for example, a reactor or a section of a reactor in which the reaction conditions may be controlled independently from the rest of the reactor. Typically, the reaction zones are reactors.

The number of separation zones is not critical, but typically there would be between 1 and 10 separation zones. By the term “separation zone” as used herein, it is meant a zone in which a product stream is separated from the process stream. The means for separating the product stream from the process stream is not critical to the present invention. Typically, the means for separating the product stream from the process stream includes a means selected from: decantation; pervaporation; distillation; flashing; two-phase separation; filtration; membrane separation; settling; falling-film separation; wiped-film separation; and centrifugation. Preferably, the means for separating the product stream from the process stream comprises at least one means selected from: decantation; distillation; flashing; two-phase separation; falling-film separation; wiped-film separation; or settling.

The chemical product outlet provides a means to collect the product stream which is separated from the process stream in the at least one separation zone.

The optional waste stream outlet may be used to remove at least a portion of the process stream that has had the product stream separated from it. When the catalytic chemical reaction is performed in a batch process manner, the waste stream outlet is used to collect the process stream which has had the product stream separated from it. Said waste stream may optionally be subjected to further separation treatments in order to remove catalyst components, unused chemical reactants, any residual chemical reaction product components and/or solvent compounds.

The optional recycle loop is used to recycle at least a portion of the process stream which has had the product stream separated from it back to the at least one reaction zone, said portion of the process stream typically comprising at least one organic compound, active homogeneous catalyst and catalyst degradation products. If the catalytic chemical reaction is performed in a continuous manner wherein at least a portion of the process stream which has had the product separated from it is recycled back to the at least one reaction zone, a waste stream outlet may be used to collect at least another portion of the process stream which has had the product stream separated from it in order to reduce the build up of any chemical by-products and/or catalyst degradation products in the process stream.

In the case of hydroformylation, it is generally preferred to operate the process in a continuous manner and to recycle at least a portion of the process stream which has had the product stream separated from it back to the at least one reaction zone.

The number of magnetic filters and the position of the magnetic filters in the chemical reactor series is not critical and they can be employed at any point in the reactor system. The number of magnetic filters employed in the chemical reactor series is preferably in the range of from 1 to 100, more preferably in the range of from 1 to 60, and most preferably in the range of from 1 to 40.

In one embodiment, the process stream is split into two or more parallel process streams and a magnetic filter is applied to two or more of the parallel process streams. The implementation of this requires the process stream being split into two or more parallel process streams at one point in the chemical reactor series, at least one magnetic filter being employed in each of the parallel process streams, and then all the parallel process streams converging after magnetic filtration has been applied to each of the parallel process streams. The advantage of using more than one magnetic filter on parallel process streams at the same point in the chemical reactor series is that one of the parallel streams may be closed off from the rest of the chemical reactor series, allowing the process stream to bypass the closed-off magnetic filter via the parallel process stream, thus enabling the magnetic filter in the closed off parallel stream to be cleaned and/or maintained without having to shut down the whole chemical reactor series.

The position of the one or more magnetic filters in the reactor series may vary. Typically, the magnetic filters can be applied at one or more of the following locations described below.

Magnetic filters may be employed before any pump in the reactor series. Since wear is caused to components of pumps by the action of solid catalyst degradation product, positioning at least one magnetic filter before any pump can help reduce the wear on the pump components and reduce the amount of maintenance required for the pumps in the reactor series.

A magnetic filter may be employed between the inlet through which the homogeneous catalyst, a precursor of the homogeneous catalyst, or the individual catalyst components, are introduced and the at least one reaction zone. Since degradation of homogeneous catalyst to solid possessing ferromagnetic properties may be promoted by the presence of solid catalyst degradation products, magnetic filters employed at this point reduces the amount of any catalyst degradation products present before the at least one reaction zone and thus help reduce the further degradation of the active homogeneous catalyst. Furthermore, since the solid catalyst degradation product may also catalyse the production of undesirable by-products, reduction of the amount of any solid catalyst degradation product present in the inlet stream would help reduce the production of said by-products under the reaction conditions present in the at least one reaction zone. These problems may be especially prominent when at least a portion of the process stream is recycled to the reaction after the product stream has been separated from it.

When more than one reaction zone is present in the reactor series, magnetic filters may be employed between reaction zones. This may remove any solid catalyst degradation product formed in the preceding reaction zone and help to reduce the further degradation of the active homogeneous catalyst to catalyst degradation product in the successive reaction zone and also may reduce the amount of by-products produced in the successive reaction zone.

Magnetic filters may be employed between the at least one reaction zone and the at least one separation zone. This may remove any solids possessing ferromagnetic properties generated by the degradation of the homogeneous catalyst in the at least one reaction zone and help to reduce the further degradation of the active homogeneous catalyst to solid catalyst degradation products in the at least one separation zone and also may reduce the amount of by-products produced in the at least one separation zone. Furthermore, depending upon the type of separation employed, this may also reduce the amount of solid catalyst degradation product being present in the product stream. As an alternative to this embodiment, or in addition to this embodiment, if more than one separation zone is present, magnetic filters may be applied between separation zones.

Magnetic filters may be employed after the at least one separation zone and before the waste outlet stream. Removal of any solids possessing ferromagnetic properties from the waste outlet stream may enable a more efficient recovery of any active homogeneous catalyst, unreacted chemical reactant, any remaining product or any solvent from the waste outlet stream, if any such recovery is desired. Alternatively, if no such recovery is desired, removal of solids possessing ferromagnetic properties may make disposal of the waste outlet stream simpler or more environmentally friendly.

When at least a portion of the process stream is recycled back to the at least one reaction zone, magnetic filters may be employed after the at the least one separation zone and before the recycle loop. This reduces the amount of solids possessing ferromagnetic properties entering the at least one reaction zone and therefore potentially reduces the amount of catalyst degradation occurring in the at least one reaction zone by reducing the promoting effect that the solid catalyst degradation product may have on the active homogeneous catalyst and also reducing the production of any by-products that may be catalysed by the presence of catalyst degradation products.

The present invention may also be used to remove solids possessing ferromagnetic properties from the product stream as well as from the process stream. Hence, according to a further aspect of the present invention, there is provided a catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream comprising at least 50 wt % organic compounds, a homogeneous catalyst based upon cobalt, nickel or iron, and a degradation product of the homogeneous catalyst comprising a solid possessing ferromagnetic properties, is separated from the process stream in at least one separation zone, and wherein one or more magnetic filters are employed to remove at least a portion of the solid possessing ferromagnetic properties from the product stream without removal or deactivation of the homogeneous catalyst present in the product stream.

Employing magnetic filters on the product stream not only removes unwanted solids containing ferromagnetic properties from the product stream and thus helps prevent damage to pumps and seals in any subsequent process performed upon the product stream, but may also help to reduce the amount of side reactions which may occur to the chemical reaction product which may be catalysed by the presence of these solid catalyst degradation products.

The chemical reaction to which the present invention may be applied is not critical. Typically, the chemical reaction is selected from hydroformylation, carbonylation, dimerisation, trimerisation, tetramerisation, oligomerisation or polymerisation of organic monomers, dimerisation, trimerisation, tetramerisation, oligomerisation or polymerisation of olefins, hydrovinylation, hydrocyanation, C—C bond formation, oxidation, epoxidation, redox, isomerisation, aromatic substitution, cross-coupling, Michael addition, hydrogenation, dehydrogenation, isomerisation, dehydration, alkoxylation, alkylation, and transalkylation. Preferably, the chemical reaction is selected from hydroformylation, carbonylation, dimerisation, trimerisation, tetramerisation, oligomerisation or polymerisation of olefins, hydrocyanation, C—C bond formation and hydrogenation.

Examples of specific embodiments of the present invention include the use of a magnetic filter in a catalytic chemical reaction process, wherein the chemical reaction process is an olefin hydroformylation process, (i.e. the hydroformylation of at least one olefinic compound), and an olefin dimerisation, trimerisation, tetramerization, oligomerisation or polymerisation process (i.e. a process for dimerising, trimerising, tetramerising, oligomerising or polymerising at least one olefinic monomer).

One specific embodiment of the present invention relates to a catalytic olefin hydroformylation process, wherein the olefin hydroformylation process is performed in a process stream comprising at least 50 wt % organic compounds, a homogeneous hydroformylation catalyst based upon cobalt, nickel or iron, and a degradation product of the homogeneous catalyst comprising a solid possessing ferromagnetic properties, wherein the hydroformylation reaction occurs in at least one reaction zone and a product stream comprising alcohols and/or aldehydes is separated from the process stream in at least one separation zone, and wherein one or more magnetic filters are employed to remove at least a portion of the solid possessing ferromagnetic properties from the process stream without removal or deactivation of the homogeneous hydroformylation catalyst present in the process stream.

In particular, this embodiment of the invention relates to a process for the hydroformylation of an olefinic compound having at least one carbon-to-carbon double bond to produce an alcohol and/or aldehyde, comprising the steps of:
(i) contacting the olefinic compound with carbon monoxide and a source of hydrogen in the presence of a homogeneous hydroformylation catalyst based on cobalt, to produce a process stream, the process stream additionally comprising a degradation product of the homogeneous catalyst comprising a solid possessing ferromagnetic properties;
(ii) separating a product stream comprising an alcohol and/or aldehyde from the process stream; and
(iii) removing at least a portion of the solid possessing ferromagnetic properties from the process stream using at least one magnetic filter.

Preferably, the homogeneous hydroformylation catalyst is based on nickel or cobalt, more preferably cobalt. Examples of cobalt-based hydroformylation catalysts include unmodified cobalt catalysts, such as those used in the oxo process, and organophosphine modified cobalt hydroformylation catalysts. In one embodiment, the homogeneous hydroformylation catalyst is an organophosphine modified cobalt hydroformylation catalyst.

The organophosphine modified cobalt hydroformylation catalyst comprises cobalt in complex combination with carbon monoxide and an organophosphine ligand. By the term “complex combination” as used herein, is meant a coordination compound formed by the union of one or more carbon monoxide with one or more cobalt atoms. In one embodiment, the organophosphine component present in the organophosphine modified cobalt hydroformylation catalyst is also in complex combination with one or more cobalt atoms in addition to the carbon monoxide. In its active form the suitable organophosphine modified cobalt hydroformylation catalyst contains one or more cobalt components.

Suitable organophosphine ligands include those having a trivalent phosphorus atom having one available or unshared pair of electrons. Any essentially organic derivative of trivalent phosphorus with the foregoing electronic configuration is a suitable ligand for the cobalt catalyst.

Organic radicals of any size and composition may be bonded to the phosphorus atom. For example the organophosphine ligand may comprise a trivalent phosphorus having aliphatic and/or cycloaliphatic and/or heterocyclic and/or aromatic radicals satisfying its three valencies. These radicals may contain a functional group such as carbonyl, carboxyl, nitro, amino, hydroxy, saturated and/or unsaturated carbon-to-carbon linkages, and saturated and/or unsaturated non-carbon-to-carbon linkages.

It is also suitable for an organic radical to satisfy more than one of the valencies of the phosphorus atom, thereby forming a heterocyclic compound with a trivalent phosphorus atom. For example, an alkylene radical may satisfy two phosphorus valencies with its two open valencies and thereby form a cyclic compound. Another example would be an alkylene dioxy radical that forms a cyclic compound where the two oxygen atoms link an alkylene radical to the phosphorus atom. In these two examples, the third phosphorus valency may be satisfied by any other organic radical.

Another type of structure involving trivalent phosphorus having an available pair of electrons is one containing a plurality of such phosphorus atoms linked by organic radicals. This type of a compound is typically called a bidentate ligand when two such phosphorus atoms are present, a tridentate ligand when three such phosphorus atoms are present, and so forth.

Suitable organophosphine modified cobalt hydroformylation catalysts and their methods of preparation are disclosed in U.S. Pat. Nos. 3,369,050, 3,501,515, 3,448,158, 3,448,157, 3,420,898 and 3,440,291, all of which are incorporated herein by reference.

The specific olefinic compound having at least one carbon-to-carbon double bond used in the specific hydroformylation embodiment of the present invention is not critical to the present invention. Preferably, the olefinic compound for the hydroformylation process of the present invention is a C₃ to C₄₀ olefin, more preferably a C₅ to C₃₀ olefin and most preferably a C₆ to C₂₀ olefin. The olefinic compound may be: acyclic or cyclic, mono-olefinic or poly-olefinic; an internal-olefin or alpha-olefin; substituted or unsubstituted. Preferably, the olefinic compound is an optionally substituted C₃ to C₄₀ mono-olefin.

If the olefinic compound having at least one olefinic carbon-to-carbon double bond is substituted, the substituent is typically inert under reaction conditions. Typically, the substituent is a hydrocarbyl group or based upon a heteroatom selected from O, N, Si, P or S, preferably O, N or Si. Examples of suitable hydrocarbyl substituents include cyclic or acyclic alkane, alkene and alkyne groups and aromatic rings, commonly, the hydrocarbyl substituents will be acyclic alkane groups. Examples of suitable substituents based upon a heteroatom include alcohol groups, amine groups, silane groups and the like.

The source of hydrogen in the hydroformylation process can be any hydrogen source used in the art for hydroformylation processes. Typically, the hydrogen source in the hydroformylation process is selected from acids, water, hydrogen (H₂, either gaseous hydrogen or dissolved hydrogen gas) or any combination thereof. Preferably, the source of hydrogen for the hydroformylation process is selected from water and/or gaseous hydrogen. More preferably, the source of hydrogen is gaseous hydrogen.

The process stream for the hydroformylation embodiment comprises at least the homogeneous hydroformylation catalyst, the olefinic compound, the product alcohol and/or aldehyde, carbon monoxide and hydrogen. The product stream comprises at least the product alcohol and/or aldehyde.

Admixtures of promoters and/or stabilizers and the like may also be included in the hydroformylation process of the present invention. Thus, minor amounts of phenolic stabilizers such as hydroquinone and/or alkaline agents such as hydroxides of alkali metals, for example NaOH and KOH, may be added to the reaction zone.

The olefin hydroformylation process may be performed under any known hydroformylation reaction conditions. The specific reaction conditions for the hydroformylation process are not critical and would be known to the person skilled in the art of hydroformylation.

Typically, the hydroformylation process may be performed at pressures in the range of from about 1×10⁵ Pa up to about 2×10⁸ Pa or higher. The specific pressure used is governed to some extent by the specific charge and catalyst employed. In general, pressures in the range of from about 2×10⁶ Pa to 10×10⁶ Pa and particularly in the range of from about 2.7×10⁶ Pa to about 9×10⁶ Pa are preferred.

Typically, the hydroformylation process may be performed at temperatures in the range of from about 0° C. up to about 300° C. The specific temperature used is governed to some extent by the specific charge and catalyst employed. In general, temperatures in the range of from about 80° C. to about 250° C., particularly in the range of from about 120° C. to about 240° C., are preferred.

The ratio of catalyst to the olefinic compound to be hydroformylated is generally not critical and may vary widely. It may be varied to achieve a substantially homogeneous reaction mixture. Solvents are therefore not required. However, the use of solvents which are inert, or which do not interfere to any substantial degree with the desired hydroformylation reaction under the conditions employed, may be used. Saturated liquid hydrocarbons, for example, may be used as solvent in the process, as well as alcohols, ethers, acetonitrile, sulfolane, and the like. The molar ratio of catalyst to the olefinic compound in the reaction zone at any given instant is typically at least about 1:1000000, preferably at least about 1:10000, and more preferably at least about 1:1000, and preferably at most about 10:1. A higher or lower ratio of catalyst to olefinic compound may, however, be used, but in general it is less than 1:1.

The total molar ratio of the source of hydrogen to carbon monoxide may vary widely. In general, a mole ratio of at least about 1:1, hydrogen to carbon monoxide, based upon H₂ being used as the source of hydrogen, is employed. Suitably, ratios of hydrogen to carbon monoxide, based upon H₂ being used as the source of hydrogen, comprise those within the range of from about 1:1 to about 10:1. Higher or lower ratios may, however, be employed.

The ratio of hydrogen to carbon monoxide employed is governed to some extent by the nature of the reaction product desired. If conditions are selected that will result primarily in an aldehyde product, only about one mole of hydrogen per mole of carbon monoxide (based upon H₂ being used as the source of hydrogen) reacts with the olefinic compound. When an alcohol is the preferred product of the process of the present invention, about two moles of hydrogen and about one mole of carbon monoxide (based upon H₂ being used as the source of hydrogen) react with each mole of olefinic compound.

Another specific embodiment of the present invention relates to a catalytic olefin oligomerisation process, wherein the olefin oligomerisation process is performed in a process stream comprising at least 50 wt % organic compounds, a homogeneous olefin oligomerisation catalyst based upon cobalt, nickel or iron, and a degradation product of the homogeneous catalyst comprising a solid possessing ferromagnetic properties, wherein the olefin oligomerisation process occurs in at least one reaction zone and a product stream comprising olefin oligomers is separated from the process stream in at least one separation zone, and wherein one or more magnetic filters are employed to remove at least a portion of the solid possessing ferromagnetic properties from the process stream without removal or deactivation of the homogeneous olefin oligomerisation catalyst present in the process stream.

In particular, this embodiment of the invention relates to a process for the oligomerisation of at least one olefinic monomer to produce an olefinic oligomer having at least one carbon-to-carbon double bond, comprising the steps of:
(i) contacting at least one olefinic monomer with a homogeneous oligomerisation catalyst based on cobalt, nickel or iron, to produce a process stream, the process stream additionally comprising a degradation product of the homogeneous catalyst comprising a solid possessing ferromagnetic properties;
(ii) separating a product stream comprising an olefinic oligomer from the process stream; and
(iii) removing at least a portion of the solid possessing ferromagnetic properties from the process stream using at least one magnetic filter.

Preferably, for the specific olefin oligomerisation process embodiment of the present invention, the homogeneous oligomerisation catalyst is based on nickel or iron. Any nickel or iron oligomerisation catalyst known in the art may be used, provided the active catalyst is homogeneous in the process stream.

The particular nickel-based homogeneous oligomerisation catalyst that can be used in the above process is not critical to the present invention. Any nickel based homogeneous oligomerisation catalyst known in the art may be used in the present invention. Non-limiting illustrative examples of nickel based oligomerisation catalyst are described in published patent applications WO 02/083750 and U.S. Pat. No. 3,676,523.

Typically, nickel oligomerisation catalyst compositions comprise a divalent nickel salt, a boron hydride and an organophosphorus compound.

The particular iron based homogeneous oligomerisation catalyst that can be used in the above process is not critical to the present invention. Any iron based homogeneous oligomerisation catalyst known in the art may be used in the present invention.

One type of iron based oligomerisation catalyst is based on iron complexes of the 2,6-bis(arylimino)pyridine type. Said complexes are known in the art and are described in published patent applications WO 02/00339, WO 02/12151, WO 02/06192, WO 02/28805, WO 01/58874, WO 99/02472 and WO 2005/090371.

The olefinic monomer for the oligomerisation embodiment of the present invention is not critical. Preferably, the olefinic monomer for the oligomerisation embodiment of the present invention is a C₂ to C₁₂ olefin, more preferably a C₂ to C₁₀ olefin and most preferably a C₂ to C₆ olefin. The olefin may be acyclic or cyclic; mono-olefinic or poly-olefinic; and substituted or unsubstituted. If the olefinic compound having at least one olefinic carbon-to-carbon double bond is substituted, the substituent is typically inert under reaction conditions. Typically, the substituent is a hydrocarbyl group or based upon a heteroatom selected from O, N, Si, P or S, preferably O, N or Si. Examples of suitable hydrocarbyl substituents include cyclic or acylcic alkane, alkene and alkyne groups and aromatic rings, commonly, the hydrocarbyl substituents will be acyclic alkane groups. Examples of suitable substituents based upon a heteroatom include alcohol groups, amine groups, silane groups and the like.

Preferably, the olefin is a C₂ to C₁₂ mono-olefin. In one typical embodiment, the olefin reactant for the oligomerisation process of the present invention is selected from ethylene, propylene, butylene and any combination thereof. In a preferred embodiment, the olefinic monomer is selected from ethylene, propylene and combinations thereof. Most preferably, the olefinic monomer used in the oligomerisation process of the present invention is ethylene.

The process stream for the oligomerisation process typically comprises at least the homogeneous oligomerisation catalyst, the olefinic monomer, and the olefinic oligomer. Other compounds such as stabilisers and/or promoters may be included in the process stream of the oligomerisation process of the present invention. The product stream comprises at least the olefinic oligomer.

The olefin oligomerisation process may be performed under any known oligomerisation reaction conditions. The specific reaction conditions for the oligomerisation process are not critical and would be known to the person skilled in the art of oligomerisation.

Typically, the temperature used in the oligomerisation process is in the range of from −100° C. to +300° C., preferably in the range of from 0° C. to 200° C., and more preferably in the range of from 50° C. to 150° C.

The oligomerisation reaction may be conveniently carried out at a pressure of from 1×10⁴ Pa to 1.5×10⁷ Pa, more preferably 1×10⁶ Pa to 1×10⁷ Pa, and most preferably 1.5×10⁶ Pa to 5×10⁶ Pa.

The optimum conditions of temperature and pressure used for a particular catalyst system to maximise the yield of oligomer, and to minimise the competing reactions such as dimerisation and polymerisation can be readily established by one skilled in the art.

The present invention will now be illustrated by the following non-limiting examples.

### EXAMPLES

The examples are performed using a pilot plant comprising a reactor zone which contains four individual reactors, each of 2 litre in volume, connected in series. A continuous stream of olefin feedstock (280 g/hr) (NEODENE-1112 or NEODENE-1314 (NEODENE is a trademark)), from Shell Chemical Company, catalyst components (cobalt octoate and an organophosphine ligand (9-eicosyl-9-phosphabicyclononane)), fresh syngas (ratio of H₂/CO in the range of 1.5 to 2.5) and recycled catalyst, is fed to the first reactor. The pressure of the first reactor is maintained at 5×10⁶ Pa. The temperatures of the reactors are maintained in the range of from 170 to 220° C.

After depressurisation, the product alcohols (formed by hydroformylation of the olefin feed stream) and a heavy-bottoms stream comprising heavy by-products with the catalyst dissolved therein, are separated via a short-path distillation. The heavy-bottom stream containing the homogeneous cobalt catalyst is recycled back to the first reactor. The experiment is carried out in a continuous mode.

Feed rates of catalyst components are adjusted to maintain the targeted catalyst concentration and composition: 0.25 wt % cobalt, organophosphine ligand/Co in the range of 1.0 to 1.5.

All of the examples are performed using the following solutions of catalyst components: 10% wt of Co(octoate)₂ dissolved in the respective product alcohol, 7.5% wt of an organophosphine ligand dissolved in the respective olefin feedstock solution. The respective product alcohol used is the alcohol composition formed by the hydroformylation of the olefin feedstock of the example.

### Example 1 (Comparative)

Operating in a continuous mode as above, without magnetic filtration, over a ten-month period, the gear-pump (located just after the short-path distiller used for transferring the heavy-bottom stream containing the cobalt catalyst back to the first reactor), fails on an average in the range of every 7 to 21 days of operation. The failure of the gear-pump is due to excessive wear and solid catalyst fouling between the gears of the pump by the solid catalyst degradation products.

### Example 2

Operating in a continuous mode as above, except that magnetic filtration is applied in the form of between 4 and 6 magnetic balls (6 mm in diameter, K-06-C from SuperMagnete.de) being held in a cage assembly positioned within the pipe-work just upstream of the gear-pump. After every 1-4 wks of operation, the, now fouled, magnetic balls are replaced for clean magnetic balls. Over a ten-month period of operation using the magnetic filtration, the gear-pump does not fail.

1. A catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream is separated from the process stream in at least one separation zone, and wherein one or more magnetic filters are employed to remove at least a portion of the solid possessing ferromagnetic properties from the process stream without removal or deactivation of the homogeneous catalyst present in the process stream.

2. A catalytic chemical reaction process according to claim 1, wherein the process is a continuous process and at least part of the process stream is recycled back into the reactor via at least one recycle loop.

3. A catalytic chemical reaction process according to claim 1 or 2, wherein at least one magnetic filter is situated after the at least one reaction zone and before at least one of the separation zones.

4. A catalytic chemical reaction process according to any one of claims 1 to 3, wherein at least one magnetic filter is positioned after at least one of the separation zones.

5. A catalytic chemical reaction process according to any one of claims 2 to 4, wherein at least one magnetic filter is positioned in the recycle loop.

6. A catalytic chemical reaction process, wherein the chemical reaction process is performed in a process stream comprising at least 50 wt % organic compounds, a homogeneous catalyst based upon cobalt, nickel or iron, and a degradation product of the homogeneous catalyst comprising a solid possessing ferromagnetic properties, wherein the chemical reaction occurs in at least one reaction zone and a product stream comprising at least 50 wt % organic compounds, a homogeneous catalyst based upon cobalt, nickel or iron, and a degradation product of the homogeneous catalyst comprising a solid possessing ferromagnetic properties, is separated from the process stream in at least one separation zone, and wherein one or more magnetic filters are employed to remove at least a portion of the solid possessing ferromagnetic properties from the product stream without removal or deactivation of the homogeneous catalyst present in the product stream.

7. A catalytic chemical reaction process according to any one of claims 1 to 6, wherein the catalytic chemical reaction process is an olefin hydroformylation process.

8. A catalytic chemical reaction process according to any one of claims 1 to 6, wherein the catalytic chemical reaction process is an olefin oligomerisation process.

9. Process for the hydroformylation of an olefinic compound having at least one carbon-to-carbon double bond to produce an alcohol and/or aldehyde, comprising the steps of:
(i) contacting the olefinic compound with carbon monoxide and a source of hydrogen in the presence of a homogeneous hydroformylation catalyst based on cobalt, to produce a process stream, the process stream additionally comprising a degradation product of the homogeneous catalyst comprising a solid possessing ferromagnetic properties;
(ii) separating a product stream comprising an alcohol and/or aldehyde from the process stream; and
(iii) removing at least a portion of the solid possessing ferromagnetic properties from the process stream using at least one magnetic filter.

10. Process for the oligomerisation of at least one olefinic monomer to produce an olefinic oligomer having at least one carbon-to-carbon double bond, comprising the steps of:
(i) contacting at least one olefinic monomer with a homogeneous oligomerisation catalyst based on cobalt, nickel or iron, to produce a process stream, the process stream additionally comprising a degradation product of the homogeneous catalyst comprising a solid possessing ferromagnetic properties;
(ii) separating a product stream comprising an olefinic oligomer from the process stream; and
(iii) removing at least a portion of the solid possessing ferromagnetic properties from the process stream using at least one magnetic filter.

11. The use of a magnetic filter in a homogeneous catalytic chemical reaction process to remove at least a portion of solids possessing ferromagnetic properties from a process and/or product stream without removal or deactivation of the homogeneous catalyst present in the process and/or product stream.
